(19) United States
(12) Patent Application Publication
(10) Pub. No.: US 2022/0031760 A1
(43) Pub. Date: Feb. 3, 2022

(54) COMPOSITIONS AND METHODS FOR REDUCING INFLAMMATION

(71) Applicant: HISTOGEN, INC., San Diego, CA (US)

(72) Inventors: Gail K. Naughton, Encinitas, CA (US); Martin Latterich, San Marcos, CA (US); Michael Zimber, Cardiff, CA (US)

(21) Appl. No.: 17/393,356

(22) Filed: Aug. 3, 2021

Related U.S. Application Data
(60) Provisional application No. 63/060,560, filed on Aug. 3, 2020.

(51) Int. Cl. A61K 35/33; A61K 35/28; A61P 29/00; A61P 19/08

(52) U.S. Cl. CPC A61K 35/33; A61P 19/08; A61P 29/00; A61K 35/28

(57) ABSTRACT

The present invention is directed to a method of producing compositions derived from culturing cells under hypoxic conditions on a biocompatible surface in vitro. The culturing method produces both an extracellular matrix composition and a conditioned culture medium composition, which may be used separately or in combination to obtain physiologically acceptable compositions useful in a variety of medical and therapeutic applications.

### CROSS-REFERENCE TO RELATED APPLICATIONS

This application claims benefit of priority under 35 U.S.C. § 119(e) of U.S. Ser. No. 63/060,560, filed Aug. 3, 2020, the entire contents of which is incorporated herein by reference in its entirety.

### BACKGROUND OF THE INVENTION

### Field of the Invention

The present invention relates generally to the production and use of extracellular matrix and/or conditioned culture medium compositions and more specifically to compositions obtained by culturing cells under hypoxic conditions on a surface in a suitable growth medium.

### Background Information

The extracellular matrix (ECM) is a complex structural entity surrounding and supporting cells that are found in vivo within mammalian tissues. The ECM is often referred to as the connective tissue. The ECM is primarily composed of three major classes of biomolecules including structural proteins such as collagens and elastins, specialized proteins such as fibrillins, fibronectins, and laminins, and proteoglycans. Conditioned culture medium (CCM) contains biologically active components obtained from previously cultured cells or tissues that have released into the media substances affecting certain cell function. It has been found that ECM and CCM compositions derived in vitro from cells grown under hypoxic conditions have therapeutic properties beneficial for treating certain conditions.

Intervertebral disc degeneration (IDD) is a major cause of chronic and debilitating back pain whose prevalence and severity make it a major health issue of substantial socioeconomic importance. The prevalence of chronic disabling back pain has tripled over the last 20 years and the treatment of lower back pain in the United States is estimated to cost more than $100 billion per year. Current treatment options are mostly palliative since disc tissue has little intrinsic repair capacity once a matrix synthesis/degradation imbalance occurs. There is a need for new methods of treating IDD.

### SUMMARY OF THE INVENTION

The present invention is based in part on the seminal discovery that cell conditioned medium (CCM) and human extracellular matrix (hECM) have therapeutic properties for treating certain disorders such as intervertebral disc degeneration (IDD).

In one embodiment, the present invention provides methods for reducing inflammation in a subject by administering to the subject a conditioned culture medium (CCM) composition or an extracellular matrix (ECM) composition, wherein the CCM and ECM compositions are produced by culturing fibroblast cells on microcarrier beads or a three-dimensional surface under hypoxic conditions of 1-5% oxygen, thereby producing multipotent stem cells which produce a CCM and an ECM fraction, thereby reducing inflammation.

In one aspect, the inflammation is associated with bone degeneration, spinal disk degeneration, osteoarthritis, pruritis, skin inflammation, psoriasis, multiple sclerosis, rheumatoid arthritis, osteoarthritis, systemic lupus erythematosus, Hashimoto's thyroidis, myasthenia gravis, diabetes type I or II, asthma, inflammatory lung injury, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, inflammatory bowel disease, Crohn's disease, ulcerative colitis, an inflammatory disease of the joints, skin, or muscle, acute or chronic idiopathic inflammatory arthritis, myositis, a demyelinating disease, chronic obstructive pulmonary disease, interstitial lung disease, interstitial nephritis or chronic active hepatitis. In certain aspects, the inflammation is associated with bone degeneration, spinal disk degeneration or osteoarthritis.

In an additional aspect, enzyme and protease activity in the anulus fibrosus (AF) and nucleus pulposus (NP) are decreased. In some aspects, the expression of IL-1, IL-6 and/or TNF in anulus fibrosus (AF) tissue is decreased and/or the expression of ACAN, MMP3 and/or ADAMTS4 is increased in AF tissue following administration of the CCM or the ECM. In certain aspects, the expression of TNF and/or MMP3 in nucleus pulposus (NP) tissue is decreased and/or expression of ADAMTS4 and/or ACAN in NP tissue following administration of the CCM or the ECM. In a further aspect, aggrecan is produced. In one aspect, the fibroblast cells are neonatal fibroblast cells.

In an additional embodiment, the present invention provides methods of treating intervertebrate disc degeneration (IDD) in a subject comprising administering to the subject a cell conditioned media (CCM) composition and/or an extracellular matrix (ECM) composition, wherein the CCM and ECM compositions are produced by culturing fibroblast cells on microcarrier beads or a three-dimensional surface under hypoxic conditions of 1-5% oxygen, thereby producing multipotent stem cells which produce a CCM and an ECM fraction, thereby treating IDD.

In an additional aspect, enzyme and protease activity in the anulus fibrosus (AF) and nucleus pulposus (NP) are decreased. In some aspects, the expression of IL-1, IL-6 and/or TNF in anulus fibrosus (AF) tissue is decreased and/or the expression of ACAN, MMP3 and/or ADAMTS4 is increased in AF tissue following administration of the CCM or the ECM. In certain aspects, the expression of TNF and/or MMP3 in nucleus pulposus (NP) tissue is decreased and/or expression of ADAMTS4 and/or ACAN in NP tissue following administration of the CCM or the ECM. In one aspect, the fibroblast cells are neonatal fibroblast cells.

In one aspect, aggrecan is produced. In an additional aspect, there is an increase in disc space and/or disc height. In a further aspect, there is a decrease in disc degeneration. In another aspect, hyaline cartilage and/or fibrous tissue is regenerated in the disc.

In a further embodiment, the present invention provides an anti-inflammatory composition which is a CCM composition or an ECM composition produced by culturing fibroblast cells on microcarrier beads or a three-dimensional surface under hypoxic conditions of 1-5% oxygen, thereby producing multipotent stem cells which produce a soluble and a non-soluble fraction and a pharmaceutically acceptable carrier.

### BRIEF DESCRIPTION OF THE DRAWINGS

FIG. 1 shows how disc height index is calculated.

FIGS. 2A-2B show the proliferation of cells treated with a CCM composition. FIG. 2A: a graph that shows the proliferation of cells treated with a CCM and untreated cells. FIG. 2B: shows the morphology of cells treated with CCM and untreated cells.

FIGS. 3A-3B show the effect of CCM and ECM on gene expression in annulus fibrosus (AF) and nucleus pulposus (NP). FIG. 3A: shows the gene expression of IL-1, IL-6, TNF, MMP3, ADAMTS4, ACAN and COL2A in AF tissue. FIG. 3B: shows the gene expression of IL-1, IL-6, TNF, MMP3, ADAMTS4, ACAN and COL2A in NP tissue.

FIG. 4 shows X-ray images of discs 4 weeks post-injection of PBS, hECM and CCM.

FIG. 5 shows 7T MRI images 4 weeks post-injection of PBS, hECM and CCM.

FIG. 6 shows the Pfirrmann grading of PBS, hECM and CCM treated discs at 4 weeks.

FIGS. 7A-7B show histological analysis discs post injection with PBS, hECM and CCM. FIG. 7A: graph of the histology scores of the discs. FIG. 7B: histology analysis of AF and NP post-injection with PBS, hECM and CCM.

FIG. 8 shows NPCs proliferating in a dose-dependent manner upon treatment with a cCCM formulation.

FIG. 9 shows the proliferative effect of different recombinant human growth factors and cCCM in the NPC assay.

### DETAILED DESCRIPTION OF THE INVENTION

The present invention is based in part on the seminal discovery that cell conditioned medium (CCM) and human extracellular matrix (hECM) have therapeutic properties for treating certain disorders such as intervertebral disc degeneration (IDD).

Intervertebral discs (IVDs) are composed of an annulus fibrosus (AF) and a nucleus pulposus (NP). The AF encloses the NP and is a strong radial tire-like structure made up of concentric sheets of collagen fibers connected to the vertebral endplates. The sheets are orientated at various angles. Both the AF and NP are composed of water, collagen, and proteoglycans (PGs). The NP contains a hydrated gel-like matter that resists compression. In early disc degeneration, changes to the matrix of the NP and inner AF are believed to be a result of a shift in the balance of anabolic and catabolic activities to result in an increased proteolytic activity. Re-search has shown that an onset of an inflammatory process plays a major role in mediating the matrix breakdown. Disc cells are capable of producing several proinflammatory cytokines, such as interleukin (IL)-1, IL-4, IL-6, IL-8, IL-112, IL-17, interferon-γ, and tumor necrosis factor-α. These cytokines mimic noxious effects and are powerful direct stimuli of nociceptive triggers. In addition, by-products of disc cell metabolism, such as lactic acid, may also be noxious. It is believed that such noxious stimuli irritate the nociceptive receptors in the outer AF and osseous endplates. Inner and outer AF cells produce proinflammatory cytokines IL-1 and TNF-α which have shown to be directly involved in pain signaling by sensitization. TNF-α has been shown to be higher and more strongly correlated to disc degeneration from symptomatic as compared to asymptomatic patients having similar amounts of degeneration. In moderate to advanced intervertebral disc degeneration (IDD) a disorganization of the AF and general fibrosis of the NP occurs which contribute to the AF becoming stiffer and weaker with age, and the development of AF tears.

IVDs contain very few cells embedded in a matrix rich in collagens, glycoproteins, and proteoglycans. Healthy cells maintain and re-pair the matrix, however damaged IVDs have an imbalance between matrix synthesis and secretion. The degraded matrix can no longer carry the load effectively and the cells become necrotic as disc degeneration continues. Replacing the proper matrix composition while reducing inflammation and protease activity has the potential of reversing the disc degenerative process and regenerating a functional IVD.

The collagen network is similar to that in hyaline cartilage and increases from the center of the NP to the outer AF, providing a network to support the cells and confine the proteoglycans. Hydrated proteoglycans are immobilized within and inflate the collagen network. The AF contains collagen type I, II, III, V, VI, IX, XI and the NP contains type II, VI, IX, and XI. The concentration of the proteoglycans in the IVD plays a major role in providing the correct mechanical properties and the hydrophilicity of the proteoglycans allows them to influence the collagen organization. The cells of the NP produce significantly more proteoglycans than in the AF, which are responsible for water attraction and retention and swelling.

Cells represent only 1% of the IVD volume with chondrocyte-like cells in the NP, fibrocartilaginous cells in the inner AF, and fibro-blast-like cells in the outer AF. These cells are specialized chondrocytes modulated by the organization of their cytoskeleton and are responsible for all of the extracellular matrix molecules in the IVD. Mechanical stress, osmotic and ionic environment, and nutrient supply are the most important stimuli of matrix secretion. Nutrients are provided to the cells by a blood supply beneath the hyaline cartilage end plate while cell activity is regulated by growth factors and cytokines. As with hyaline cartilage of the joint, nutrient transport into and through the disc is largely due to diffusion with diffusion coefficients increasing in the NP. The IVD cells are completely surrounded by a pericellular matrix (PCM) which contains collagen type IV, II and IX, aggrecan, hyaluronan, decorin, and fibronectin. Treatments for IDD include intradiscal steroid injections to reduce inflammation none of which have been associated with prolonged pain relief and fusion and removal can have significant complications associated with them. Our plan is to use soluble (CCM) and insoluble proteins (hECM) secreted by hypoxia-induced stem cells to reduce or eliminate the inflammation associated with IDD and induce disc regeneration.

The present invention is directed to a cell conditioned media (CCM) and a naturally secreted extracellular matrix (hECM) that are very embryonic in composition and have been shown to stimulate stem cells in vivo. In contrast to adults, fetal mammals are capable of regenerating injured tissues without scarring during the first two trimesters of gestation. The scar-less healing phenomenon is associated with hypoxia (1-5% oxygen), low levels of TGFβ and FGF, and a predominance of collagens III and V. The mechanism of fetal scar-less regeneration and the embryogenetic mechanisms of chondrogenesis is still not fully understood, but a deeper understanding of how recapitulating the embryonic mechanism in the treatment of disc degeneration may offer tremendous benefit in reversing the pathogenesis of this disease. Recent research utilizing gene expression analysis to compare fetal and adult genes and their implied protein regulation in response to tissue injury has shown that the most relevant events involved proteins associated with the immune response and inflammation and proteins involved in cell growth and proliferation and cartilage tissue and development. Proteins associated with the inflammatory process were found to be significantly upregulated following injury in adults but not in fetal animals; proteoglycans were upregulated only in fetal sheep post cartilage injury.

Neonatal fibroblasts are grown under hypoxia (3-5% oxygen) and suspension culture to mimic the embryonic environment. During this culture process the fibroblasts revert back to multipotent stem cells, as evidenced by the up-regulation of pluripotent stem cell-associated genes that include, SOX2, OCT4/POU5F1, NANOG and KLF4, and by the expression of stem cell-associated proteins, including nodal, brachyury, nestin and Oct4. The CCM harvested during the manufacturing process and the hECM harvested.

The Examples provide in vitro studies which demonstrate that the CCM and hECM individually support the regulation and proliferation of human mesenchymal stem cells (hMSCs), as well as cell surface markers, showing the maintenance of stemness and inducing the upregulation of aggrecan and Collagen II. These are all important components in hyaline and hyaline-like cartilage matrix regeneration which would be critical in reversing IVD degeneration and reestablishing the natural mechanical strength of the IVD. Additional studies were performed to help analyze the CCM and hECM components and their in vitro, ex vivo, and in vivo influence on chondrocytes as well as other IVD cells.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

In various embodiments, the present invention involves methods for making ECM and CCM compositions that include one or more embryonic proteins and applications thereof. In particular the compositions are generated by culturing cells under hypoxic conditions on a two-dimensional or three-dimensional surface in a suitable growth medium. The compositions are derived by growing cells on a three-dimensional framework resulting in a multi-layer cell culture system. Cells grown on a three-dimensional framework support, in accordance with the present invention, grow in multiple layers, forming a cellular matrix. Growth of the cultured cells under hypoxic conditions results in differential gene expression as the result of hypoxic culturing conditions versus traditional culture in the ECM and the CCM.

ECM is a composition of proteins and biopolymers that substantially comprise tissue that is produced by cultivation of cells. Stromal cells, such as fibroblasts, are an anchorage dependent cell type requiring growth while attached to materials and surfaces suitable for cell culture. The ECM materials produced by the cultured cells are deposited in a three-dimensional arrangement providing spaces for the formation of tissue-like structures.

The cultivation materials providing three-dimensional architectures are referred to as scaffolds. Spaces for deposition of ECM are in the form of openings within, for example woven mesh or interstitial spaces created in a compacted configuration of spherical beads, called microcarriers.

The methods described herein produce both a non-soluble ECM composition and a soluble CCM composition. The non-soluble composition includes those secreted ECM proteins and biological components that are deposited on the support or scaffold. The soluble composition includes culture media or conditioned media in which cells have been cultured and into which the cells have secreted active agent(s) and includes those proteins and biological components not deposited on the scaffold. Both compositions may be collected, and optionally further processed, and used individually or in combination in a variety of applications as described herein.

The three-dimensional support or scaffold used to culture stromal cells may be of any material and/or shape that: (a) allows cells to attach to it (or can be modified to allow cells to attach to it); and (b) allows cells to grow in more than one layer (i.e., form a three-dimensional tissue). In other embodiments, a substantially two-dimensional sheet or membrane or beads may be used to culture cells that are sufficiently three-dimensional in form.

In some aspects, the three-dimensional surface or beads may be made of materials that degrade over time under the conditions of use. Biodegradable also refers to absorbability or degradation of a compound or composition when administered in vivo or under in vitro conditions. Biodegradation may occur through the action of biological agents, either directly or indirectly. Non-limiting examples of biodegradable materials include, among others, polylactide, polyglycolide, poly(trimethylene carbonate), poly(lactide-co-glycolide) (i.e., PLGA), polyethylene terephtalate (PET), polycaprolactone, catgut suture material, collagen (e.g., equine collagen foam), polylactic acid, or hyaluronic acid. For example, these materials may be woven into a three-dimensional framework such as a collagen sponge or collagen gel.

In other aspects, where the cultures are to be maintained for long periods of time, cryopreserved, and/or where additional structural integrity is desired, the three-dimensional framework may be comprised of a nonbiodegradable material. As used herein, a nonbiodegradable material refers to a material that does not degrade or decompose significantly under the conditions in the culture medium. Exemplary nondegradable materials include, as non-limiting examples, nylon, dacron, polystyrene, polyacrylates, polyvinyls, polytetrafluoroethylenes (PTFE), expanded PTFE (ePTFE), and cellulose. An exemplary nondegrading three-dimensional framework comprises a nylon mesh, available under the tradename Nitex®, a nylon filtration mesh having an average pore size of 140 μm and an average nylon fiber diameter of 90 μm (#3-210/36, Tetko, Inc., N.Y.).

In other aspects, the beads, scaffold or framework is a combination of biodegradeable and non-biodegradeable materials. The non-biodegradable material provides stability to the three-dimensional scaffold during culturing while the biodegradeable material allows formation of interstitial spaces sufficient for generating cell networks that produce the cellular factors sufficient for therapeutic applications. The biodegradable material may be coated onto the non-biodegradable material or woven, braided or formed into a mesh. Various combinations of biodegradable and non-biodegradable materials may be used. An exemplary combination is poly(ethylene therephtalate) (PET) fabrics coated with a thin biodegradable polymer film, poly[D-L-lactic-co-glycolic acid), in order to obtain a polar structure.

In various aspects, the scaffold or framework material may be pre-treated prior to inoculation with cells to enhance cell attachment. For example, prior to inoculation with cells, nylon screens in some embodiments are treated with 0.1 M acetic acid, and incubated in polylysine, fetal bovine serum, and/or collagen to coat the nylon. Polystyrene could be similarly treated using sulfuric acid. In other embodiments, the growth of cells in the presence of the three-dimensional support framework may be further enhanced by adding to the framework or coating it with proteins (e.g., collagens, elastin fibers, reticular fibers), glycoproteins, glycosaminoglycans (e.g., heparan sulfate, chondroitin-4-sulfate, chondroitin-6-sulfate, dermatan sulfate, keratan sulfate, etc.), fibronectins, and/or glycopolymer (poly[N-p-vinylbenzyl-D-lactoamide], PVLA) in order to improve cell attachment. Treatment of the scaffold or framework is useful where the material is a poor substrate for the attachment of cells.

In one aspect, mesh is used for production of ECM. The mesh is a woven nylon 6 material in a plain weave form with approximately 100 μm openings and approximately 125 μm thick. In culture, fibroblast cells attach to the nylon through charged protein interactions and grow into the voids of the mesh while producing and depositing ECM proteins. Mesh openings that are excessively large or small may not be effective but could differ from those above without substantially altering the ability to produce or deposit ECM. In another aspect, other woven materials are used for ECM production, such as polyolefin's, in weave configurations giving adequate geometry for cell growth and ECM deposition.

In other aspects, the scaffold for generating the cultured tissues is composed of microcarriers, which are beads or particles. The beads may be microscopic or macroscopic and may further be dimensioned so as to permit penetration into tissues or compacted to form a particular geometry. In some embodiments, the framework for the cell cultures comprises particles that, in combination with the cells, form a three-dimensional tissue. The cells attach to the particles and to each other to form a three-dimensional tissue. The complex of the particles and cells is of sufficient size to be administered into tissues or organs, such as by injection or catheter. Beads or microcarriers are typically considered a two-dimensional system or scaffold.

As used herein, a “microcarriers” refers to a particle having size of nanometers to micrometers, where the particles may be any shape or geometry, being irregular, non-spherical, spherical, or ellipsoid.

The size of the microcarriers suitable for the purposes herein can be of any size suitable for the particular application. In some embodiments, the size of microcarriers suitable for the three-dimensional tissues may be those administrable by injection. In some embodiments, the microcarriers have a particle size range of at least about 1 μm, at least about 10 μm, at least about 25 μm, at least about 50 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1000 μm.

In some aspects in which the microcarriers are made of biodegradable materials. In some aspects, microcarriers comprising two or more layers of different biodegradable polymers may be used. In some embodiments, at least an outer first layer has biodegradable properties for forming the three-dimensional tissues in culture, while at least a biodegradable inner second layer, with properties different from the first layer, is made to erode when administered into a tissue or organ.

In some aspects, the microcarriers are porous microcarriers. Porous microcarriers refer to microcarriers having interstices through which molecules may diffuse in or out from the microparticle. In other embodiments, the microcarriers are non-porous microcarriers. A nonporous microparticle refers to a microparticle in which molecules of a select size do not diffuse in or out of the microparticle.

Microcarriers for use in the compositions are biocompatible and have low or no toxicity to cells. Suitable microcarriers may be chosen depending on the tissue to be treated, type of damage to be treated, the length of treatment desired, longevity of the cell culture in vivo, and time required to form the three-dimensional tissues. The microcarriers may comprise various polymers, natural or synthetic, charged (i.e., anionic or cationic) or uncharged, biodegradable, or nonbiodegradable. The polymers may be homopolymers, random copolymers, block copolymers, graft copolymers, and branched polymers.

In some aspects, the microcarriers comprise non-biodegradable microcarriers. Non-biodegradable microcapsules and microcarriers include, but not limited to, those made of polysulfones, poly(acrylonitrile-co-vinyl chloride), ethylene-vinyl acetate, hydroxyethylmethacrylate-methyl-methacrylate copolymers. These are useful to provide tissue bulking properties or in embodiments where the microcarriers are eliminated by the body.

In some aspects, the microcarriers comprise degradable scaffolds. These include microcarriers made from naturally occurring polymers, non-limiting example of which include, among others, fibrin, casein, serum albumin, collagen, gelatin, lecithin, chitosan, alginate or poly-amino acids such as poly-lysine. In other aspects, the degradable microcarriers are made of synthetic polymers, non-limiting examples of which include, among others, polylactide (PLA), polyglycolide (PGA), poly(lactide-co-gly colide) (PLGA), poly(caprolactone), polydioxanone trimethylene carbonate, polyhybroxyalkonates (e.g., poly(hydroxybutyrate), poly(ethyl glutamate), poly(DTH iminocarbony(bisphenol A iminocarbonate), poly(ortho ester), and polycyanoacrylates.

In some aspects, the microcarriers comprise hydrogels, which are typically hydrophilic polymer networks filled with water. Hydrogels have the advantage of selective trigger of polymer swelling. Depending on the composition of the polymer network, swelling of the microparticle may be triggered by a variety of stimuli, including pH, ionic strength, thermal, electrical, ultrasound, and enzyme activities. Non-limiting examples of polymers useful in hydrogel compositions include, among others, those formed from polymers of poly(lactide-co-glycolide); poly(N-isopropylacrylamide); poly(methacrylic acid-g-polyethylene glycol); polyacrylic acid and poly(oxypropylene-co-oxyethylene) glycol; and natural compounds such as chrondroitin sulfate, chitosan, gelatin, fibrinogen, or mixtures of synthetic and natural polymers, for example chitosan-poly (ethylene oxide). The polymers may be crosslinked reversibly or irreversibly to form gels adaptable for forming three-dimensional tissues.

In exemplary aspects, the microcarriers or beads for use in the present invention are composed wholly or composed partly of dextran.

In accordance with the present invention the culturing method is applicable to proliferation of different types of cells, including stromal cells, such as fibroblasts, and particularly primary human neonatal foreskin fibroblasts. In various aspects, the cells inoculated onto the scaffold or framework can be stromal cells comprising fibroblasts, with or without other cells, as further described below. In some embodiments, the cells are stromal cells that are typically derived from connective tissue, including, but not limited to: (1) bone; (2) loose connective tissue, including collagen and elastin; (3) the fibrous connective tissue that forms ligaments and tendons, (4) cartilage; (5) the ECM of blood; (6) adipose tissue, which comprises adipocytes; and (7) fibroblasts.

Stromal cells can be derived from various tissues or organs, such as skin, heart, blood vessels, bone marrow, skeletal muscle, liver, pancreas, brain, foreskin, which can be obtained by biopsy (where appropriate) or upon autopsy. In one aspect, fetal fibroblasts can be obtained in high quantity from foreskin, such as neonatal foreskins.

In some aspects, the cells comprise fibroblasts, which can be from a fetal, neonatal, adult origin, or a combination thereof. In some aspects, the stromal cells comprise fetal fibroblasts, which can support the growth of a variety of different cells and/or tissues. As used herein, a fetal fibroblast refers to fibroblasts derived from fetal sources. As used herein, neonatal fibroblast refers to fibroblasts derived from newborn sources. Under appropriate conditions, fibroblasts can give rise to other cells, such as bone cells, fat cells, and smooth muscle cells and other cells of mesodermal origin. In some embodiments, the fibroblasts comprise dermal fibroblasts, which are fibroblasts derived from skin. Normal human dermal fibroblasts can be isolated from neonatal foreskin. These cells are typically cryopreserved at the end of the primary culture.

For certain uses in vivo it is preferable to obtain the stromal cells from the patient's own tissues. The growth of cells in the presence of the three-dimensional stromal support framework can be further enhanced by adding to the framework, or coating the framework support with proteins, e.g., collagens, laminins, elastic fibers, reticular fibers, glycoproteins; glycosaminoglycans, e.g., heparin sulfate, chondroitin-4-sulfate, chondroitin-6-sulfate, dermatan sulfate, keratan sulfate, etc.; a cellular matrix, and/or other materials.

Thus, since the two-dimensional or three-dimensional culture systems described herein are suitable for growth of diverse cell types and tissues, and depending upon the tissue to be cultured and the collagen types desired, the appropriate stromal cells may be selected to inoculate the framework.

While the methods and applications of the present invention are suitable for use with different cell types, such as tissue specific cells or different types of stromal cells as discussed herein, derivation of the cells for use with the present invention may also be species specific. Accordingly, ECM compositions may be generated that are species specific. For example, the cells for use in the present invention may include human cells. For example, the cells may be human fibroblasts. Likewise, the cells are from another species of animal, such as equine (horse), canine (dog) or feline (cat) cells. Additionally, cells from one species or strain of species may be used to generate ECM compositions for use in other species or related strains (e.g., allogeneic, syngeneic and xenogeneic). It is also to be appreciated that cells derived from various species may be combined to generate multi-species ECM compositions.

Accordingly, the methods and compositions of the present invention are suitable in applications involving non-human animals. As used herein, “veterinary” refers to the medical science concerned or connected with the medical or surgical treatment of animals, especially domestic animals. Common veterinary animals may include mammals, amphibians, avians, reptiles and fishes. For example, typical mammals may include dogs, cats, horses, rabbits, primates, rodents, and farm animals, such as cows, horses, goats, sheep, and pigs.

As discussed above, additional cells may be present in the culture with the stromal cells. These additional cells may have a number of beneficial effects, including, among others, supporting long term growth in culture, enhancing synthesis of growth factors, and promoting attachment of cells to the scaffold. Additional cell types include as non-limiting examples, smooth muscle cells, cardiac muscle cells, endothelial cells, skeletal muscle cells, endothelial cells, pericytes, macrophages, monocytes, and adipocytes. Such cells may be inoculated onto the framework along with fibroblasts, or in some aspects, in the absence of fibroblasts. These stromal cells may be derived from appropriate tissues or organs, including, by way of example and not limitation, skin, heart, blood vessels, bone marrow, skeletal muscle, liver, pancreas, and brain. In other aspects, one or more other cell types, excluding fibroblasts, are inoculated onto the scaffold. In still other aspects, the scaffolds are inoculated only with fibroblast cells.

After an appropriate three-dimensional scaffold is prepared, it is inoculated by seeding with the stromal cells. Inoculation of the scaffold may be done in a variety of ways, such as sedimentation.

Incubation of the inoculated culture is performed under hypoxic conditions, which is discovered to produce an ECM and CCM with unique properties as compared to ECM and CCM generated under normal culture conditions. As used herein, hypoxic conditions are characterized by a lower oxygen concentration as compared to the oxygen concentration of ambient air (approximately 15%-20% oxygen). In one aspect, hypoxic conditions are characterized by an oxygen concentration less than about 10%. In another aspect hypoxic conditions are characterized by an oxygen concentration of about 1% to 10%, 1% to 9%, 1% to 8%, 1% to 7%, 1% to 6%, 1% to 5%, 1% to 4%, 1% to 3%, or 1% to 2%. In a certain aspect, the system maintains about 1-3% oxygen within the culture vessel. Hypoxic conditions can be created and maintained by using a culture apparatus that allows one to control ambient gas concentrations, for example, an anaerobic chamber.

Incubation of cell cultures is typically performed in normal atmosphere with 15-20% oxygen and 5% CO₂ for expansion and seeding, at which point low oxygen cultures are split to an airtight chamber that is flooded with 95% nitrogen/5% CO₂ so that a hypoxic environment is created within the culture medium.

For example, cell cultures for producing ECM under hypoxic conditions are initially grown in incubation at 37° C. and 95% air/5% CO₂ for 2-3 weeks. Following the period of near atmospheric cultivation, the cell cultures are incubated in a chamber designed for anaerobic cultivation that is purged with a gas mixture of approximately 95% nitrogen and 5% CO₂. Expended growth media is replaced with fresh media at atmospheric oxygen level through the culture period and after media is exchanged the mesh filled petri dishes are place in the anaerobic chamber, the chamber is purged with 95% nitrogen/5% CO₂ then incubated at 37° C. Cultured mesh are harvested when they reach the desired size or contain the desire biological components. Cells are cultured under hypoxic conditions for at least 2, 3, 4, 5, 6, 7, 8, 10, 12, 14, 16, 18, 20, 22 or 24 weeks.

During the incubation period, the stromal cells will grow linearly along and envelop the three-dimensional framework before beginning to grow into the openings of the framework. The growing cells produce a myriad of growth factors, regulatory factors and proteins, some of which are secreted in the surrounding media, and others that are deposited on the support to make up the ECM more fully discussed below. Growth and regulatory factors can be added to the culture, but are not necessary. Culture of the stromal cells produces both non-soluble and soluble fractions. The cells are grown to an appropriate degree to allow for adequate deposition of ECM proteins.

During culturing of the three-dimensional tissues, proliferating cells may be released from the framework and stick to the walls of the culture vessel where they may continue to proliferate and form a confluent monolayer. To minimize this occurrence, which may affect the growth of cells, released cells may be removed during feeding or by transferring the three-dimensional cell culture to a new culture vessel. Removal of the confluent monolayer or transfer of the cultured tissue to fresh media in a new vessel maintains or restores proliferative activity of the three-dimensional cultures. In some aspects, removal or transfers may be done in a culture vessel which has a monolayer of cultured cells exceeding 25% confluency. Alternatively, the culture in some embodiments is agitated to prevent the released cells from sticking; in others, fresh media is infused continuously through the system. In some aspects, two or more cell types can be cultured together either at the same time or one first followed by the second (e.g., fibroblasts and smooth muscle cells or endothelial cells).

After inoculation of the three-dimensional scaffolds, the cell culture is incubated in an appropriate nutrient medium and incubation conditions that supports growth of cells into the three-dimensional tissues. Many commercially available media such as Dulbecco's Modified Eagles Medium (DMEM), RPMI 1640, Fisher's, Iscove's, and McCoy's, may be suitable for supporting the growth of the cell cultures. The medium may be supplemented with additional salts, carbon sources, amino acids, serum and serum components, vitamins, minerals, reducing agents, buffering agents, lipids, nucleosides, antibiotics, attachment factors, and growth factors. Formulations for different types of culture media are described in various reference works available to the skilled artisan (e.g., Methods for Preparation of Media, Supplements and Substrates for Serum Free Animal Cell Cultures, Alan R. Liss, New York (1984); Tissue Culture: Laboratory Procedures, John Wiley & Sons, Chichester, England (1996); Culture of Animal Cells, A Manual of Basic Techniques, 4 th Ed., Wiley-Liss (2000)).

The growth or culture media used in any of the culturing steps of the present invention, whether under aerobic or hypoxic conditions, may include serum, or be serum free. In one aspect, the media is Dulbecco's Modified Eagle Medium with 4.5 g/L glucose, alanyl-L-glutamine, Eq 2 mM, and nominally supplemented with 10% fetal bovine serum. In another aspect, the media is a serum free media and is Dulbecco's Modified Eagle Medium with 4.5 g/L glucose base medium with Glutamax®, supplemented with 0.5% serum albumin, 2 μg/ml heparin, 1 μg/ml recombinant basic FGF, 1 μg/ml soybean trypsin inhibitor, 1× ITS supplement (insulin-transferrin-selenium, Sigma Cat. No. 13146), 1:1000 diluted fatty acid supplement (Sigma Cat. No. 7050), and 1:1000 diluted cholesterol. Additionally, the same media can be used for both hypoxic and aerobic cultivation. In one aspect, the growth media is changed from serum based media to serum free media after seeding and the first week of growth.

Incubation conditions will be under appropriate conditions of pH, temperature, and gas (e.g., O₂, CO₂, etc.) to maintain a hypoxic growth condition. In some embodiments, the three-dimensional cell culture can be suspended in the medium during the incubation period in order to maximize proliferative activity and generate factors that facilitate the desired biological activities of the fractions. In addition, the culture may be “fed” periodically to remove the spent media, depopulate released cells, and add new nutrient source. During the incubation period, the cultured cells grow linearly along and envelop the filaments of the three-dimensional scaffold before beginning to grow into the openings of the scaffold.

In another embodiment, the invention includes generation of a stem cell by culturing cells under hypoxic conditions (e.g., fibroblasts, under hypoxic conditions) thereby generating cells that express genes characteristics of stem cells at a level at least 3 fold greater than when grown under normoxic conditions. Such genes may include Oct4, Sox2, KLF4, NANOG and cMyc, for example.

During incubation under hypoxic conditions, as compared to incubation under normal atmospheric oxygen concentrations of about 15-20%, thousands of genes are differentially expressed. Several genes have been found to be upregulated or downregulated in such compositions, most notably certain laminin species, collagen species and Wnt factors. In various aspects, the three-dimensional ECM may be defined by the characteristic fingerprint or suite of cellular products produced by the cells due to growth in hypoxic condition as compared with growth under normal conditions. In the ECM compositions specifically exemplified herein, the three-dimensional tissues and surrounding media are characterized by expression and/or secretion of various factors.

The three-dimensional tissues and compositions described herein have ECM that is present on the scaffold or framework. In some aspects, the ECM includes various laminin and collagen types due to growth under hypoxic conditions and selection of cells grown on the support. The proportions of ECM proteins deposited can be manipulated or enhanced by selecting fibroblasts which elaborate the appropriate collagen type as well as growing the cells under hypoxic conditions in which expression of specific laminin and collagen species are upregulated or downregulated.

Selection of fibroblasts can be accomplished in some aspects using monoclonal antibodies of an appropriate isotype or subclass that define particular collagen types. In other aspects, solid substrates, such as magnetic beads, may be used to select or eliminate cells that have bound antibody. Combination of these antibodies can be used to select (positively or negatively) the fibroblasts which express the desired collagen type. Alternatively, the stroma used to inoculate the framework can be a mixture of cells which synthesize the desired collagen types. The distribution and origins of the exemplary type of collagen are shown in Table 1.

TABLE 1
Distributions and Origins of Various Types of Collagen

| | Principle Tissue | |
|---|---|---|
| Collagen Type | Distribution | Cells of Origin |
| I | Loose and dense ordinary | Fibroblasts and reticular |
| | connective tissue; collagen | cells; smooth muscle cells |
| | fibers | |
| | Fibrocartilage | |
| | Bone | Osteoblasts |
| | Dentin | Odontoblasts |
| II | Hyaline and elastic | Chondrocytes |
| | cartilage | |
| | Vitreous body of eye | Retinal cells |
| III | Loose connective tissue; | Fibroblasts and reticular |
| | reticular fibers | cells |
| | Papillary layer of dermis | Smooth muscle cells; |
| | | endothelial cells |
| | Blood vessels | |
| IV | Basement membranes | Epithelial and endothelial |
| | | cells |
| | Lens capsule of eve | Lens fibers |
| V | Fetal membranes; placenta | Fibroblasts |
| | Basement membranes | |
| | Bone | |
| | Smooth muscle | Smooth muscle cells |
| IV | Basement membranes | Epithelial and endothelial |
| | | cells |
| | Lens capsule of the eve | Lens fiber |
| V | Fetal membranes; placenta | Fibroblasts |
| | Basement membranes | |
| | Bone | |
| | Smooth muscle | Smooth muscle cells |
| VI | Connective tissue | Fibroblasts |
| VII | Epithelial basement | Fibroblasts |
| | membranes | |
| | anchoring fibrils | keratinocytes |
| VIII | Cornea | Corneal fibroblasts |
| IX | Cartilage | |
| X | Hypertrophic cartilage | |
| XI | Cartilage | |
| XII | Papillary dermis | Fibroblasts |
| XIV (undulin) | Reticular dermis | Fibroblasts |
| XVII | P170 bullous pemphigoid | Keratinocytes |
| | antigen | |

Additional types of collagen that may be present in ECM compositions are shown in Table 2.

TABLE 2
Types of Collagen and Corresponding Gene(s)

| Collagen Type | Gene(s) |
|---|---|
| I | COL1A1, COL1A2 |
| II | COL2A1 |
| III | COL3A1 |
| IV | COL4A1, COL4A2, COL4A3, |
| | COL4A4, COL4A5, COL4A6 |
| V | COL5A1, COL5A2, COLSA3 |
| VI | COL6A1, COL6A2, COL6A3 |
| VII | COL7A1 |
| VIII | COL8A1, COL8A2 |
| IX | COL9A1, COL9A2, COL9A3 |
| X | COL10A1 |
| XI | COL11A1, COL11A2 |
| XII | COL12A1 |
| XIII | COL13A1 |
| XIV | COL14A1 |
| XV | COL15A1 |
| XVI | COL16A1 |
| XVII | COL17A1 |
| XVIII | COL18A1 |
| XIX | COL19A1 |
| XX | COL20A1 |
| XXI | COL21A1 |
| XXII | COL22A1 |
| XXIII | COL23A1 |
| XXIV | COL24A1 |
| XXV | COL25A1 |
| XXVI | EMID2 |
| XXVII | COL27A1 |
| XXVIII | COL28A1 |

As discussed above the ECM and CCM compositions described herein include various collagens. As shown in Table 3 of Example 1, expression of several species of collagen are found to be upregulated in hypoxic cultured ECM and CCM compositions. Accordingly, in one aspect of the present invention, the ECM and CCM compositions including one or more embryonic proteins, includes upregulation of collagen species as compared with that produced in oxygen conditions of about 15-20% oxygen. In another aspect, the upregulated collagen species are type V alpha 1; IX alpha 1; IX alpha 2; VI alpha 2; VIII alpha 1; IV, alpha 5; VII alpha 1; XVIII alpha 1; and XII alpha 1.

In addition to various collagens, the ECM and CCMs composition described herein include various laminins. Laminins are a family of glycoprotein heterotrimers composed of an alpha, beta, and gamma chain subunit joined together through a coiled-coil domain. To date, 5 alpha, 4 beta, and 3 gamma laminin chains have been identified that can combine to form 15 different isoforms. Within this structure are identifiable domains that possess binding activity towards other laminin and basal lamina molecules, and membrane-bound receptors. Domains VI, IVb, and IVa form globular structures, and domains V, IIIb, and IIIa (which contain cysteine-rich EGF-like elements) form rod-like structures. Domains I and II of the three chains participate in the formation of a triple-stranded coiled-coil structure (the long arm).

Laminin chains possess shared and unique functions and are expressed with specific temporal (developmental) and spatial (tissue-site specific) patterns. The laminin alpha-chains are considered to be the functionally important portion of the heterotrimers, as they exhibit tissue-specific distribution patterns and contain the major cell interaction sites. Vascular endothelium is known to express two laminin isoforms, with varied expression depending on the developmental stage, vessel type, and the activation state of the endothelium.

Accordingly, in one aspect of the present invention, the ECM composition including one or more embryonic proteins, includes upregulation or downregulation of various laminin species as compared with that produced in oxygen conditions of about 15-20% oxygen.

The ECM and CCM compositions described herein can include various Wnt factors. Wnt family factors are signaling molecules having roles in a myriad of cellular pathways and cell-cell interaction processes. Wnt signaling has been implicated in tumorigenesis, early mesodermal patterning of the embryo, morphogenesis of the brain and kidneys, regulation of mammary gland proliferation, and Alzheimer's disease. Expression of several species of Wnt proteins have been found to be upregulated in hypoxic cultured ECM compositions. Accordingly, in one aspect of the present invention, the ECM composition including one or more embryonic proteins, includes upregulation of Wnt species as compared with that produced in oxygen conditions of about 15-20% oxygen. In another aspect, the upregulated Wnt species are Wnt 7a and Wnt 11. In another aspect, Wnt factors produced by the three-dimensional tissues of the present invention, include at least Wnt7a, and Wnt11, which defines a characteristic or signature of the Wnt proteins present in the composition.

The culturing methods described herein, including culture under hypoxic conditions, have also been shown to upregulate expression of various growth factors. Accordingly, the ECM and CCM compositions described herein can include various growth factors, such as a vascular endothelial growth factor (VEGF). As used herein, a VEGF in intended to include all known VEGF family members. VEGFs are a sub-family of growth factors, more specifically of platelet-derived growth factor family of cystine-knot growth factors. VEGFs have a well known role in both vasculogenesis and angiogenesis. Several VEGFs are known, including VEGF-A, which was formerly known as VEGF before the discovery of other VEGF species. Other VEGF species include placenta growth factor (PlGF), VEGF-B, VEGF-C and VEGF-D. Additionally, several isoforms of human VEGF are well known.

A discussed throughout, the compositions of the present invention includes both ECM and CCM fractions or any portion thereof. It is to be understood that the compositions of the present invention may include either or both fractions, as well as any combination thereof. Additionally, individual components may be isolated from the fractions to be used individually or in combination with other isolates or known compositions.

Accordingly, in various aspects, ECM and CCM compositions produced using the methods of the present invention may be used directly or processed in various ways, the methods of which may be applicable to both the ECM and CCM compositions. The CCM, including the cell-free supernatant and media, may be subject to lyophilization for preserving and/or concentrating the factors. Various biocompatible preservatives, cryoprotectives, and stabilizer agents may be used to preserve activity where required. Examples of biocompatible agents include, among others, glycerol, dimethyl sulfoxide, and trehalose. The lyophilizate may also have one or more excipients such as buffers, bulking agents, and tonicity modifiers. The freeze-dried media may be reconstituted by addition of a suitable solution or pharmaceutical diluent, as further described below.

In other aspects, the CCM is dialyzed. Dialysis is one of the most commonly used techniques to separate sample components based on selective diffusion across a porous membrane. The pore size determines molecular-weight cutoff (MWCO) of the membrane that is characterized by the molecular-weight at which 90% of the solute is retained by the membrane. In certain aspects membranes with any pore size is contemplated depending on the desired cutoff Typical cutoffs are 5,000 Daltons, 10,000 Daltons, 30,000 Daltons, and 100,000 Daltons, however all sizes are contemplated.

In some aspects, the CCM may be processed by precipitating the active components (e.g., growth factors) in the media. Precipitation may use various procedures, such as salting out with ammonium sulfate or use of hydrophilic polymers, for example polyethylene glycol.

In other aspects, the CCM is subject to filtration using various selective filters. Processing the CCM by filtering is useful in concentrating the factors present in the fraction and also removing small molecules and solutes used in the soluble fraction. Filters with selectivity for specified molecular weights include <5000 Daltons, <10,000 Daltons, and <15,000 Daltons. Other filters may be used and the processed media assayed for therapeutic activity as described herein. Exemplary filters and concentrator system include those based on, among others, hollow fiber filters, filter disks, and filter probes (see, e.g., Amicon Stirred Ultrafiltration Cells).

In still other aspects, the CCM is subject to chromatography to remove salts, impurities, or fractionate various components of the medium. Various chromatographic techniques may be employed, such as molecular sieving, ion exchange, reverse phase, and affinity chromatographic techniques. For processing conditioned medium without significant loss of bioactivity, mild chromatographic media may be used. Non-limiting examples include, among others, dextran, agarose, polyacrylamide based separation media (e.g., available under various tradenames, such as Sephadex, Sepharose, and Sephacryl).

In still other aspects, the CCM is formulated as liposomes. The growth factors may be introduced or encapsulated into the lumen of liposomes for delivery and for extending life time of the active factors. As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which may be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids such as cholesterol; cerebrosides; sphingomyelin; glycerolipids; and other lipids (see, e.g., U.S. Pat. No. 5,833,948).

The CCM may be used directly without additional additives, or prepared as pharmaceutical compositions with various pharmaceutically acceptable excipients, vehicles or carriers. A “pharmaceutical composition” refers to a form of the soluble and/or non-soluble fractions and at least one pharmaceutically acceptable vehicle, carrier, or excipient. For intradermal, subcutaneous or intramuscular administration, the compositions may be prepared in sterile suspension, solutions or emulsions of the ECM compositions in aqueous or oily vehicles. The compositions may also contain formulating agents, such as suspending, stabilizing or dispersing agents. Formulations for injection may be presented in unit dosage form, ampules in multidose containers, with or without preservatives. Alternatively, the compositions may be presented in powder form for reconstitution with a suitable vehicle including, by way of example and not limitation, sterile pyrogen free water, saline, buffer, or dextrose solution.

In other aspects, the three-dimensional tissues are cryopreserved preparations, which are thawed prior to use. Pharmaceutically acceptable cryopreservatives include, among others, glycerol, saccharides, polyols, methylcellulose, and dimethyl sulfoxide. Saccharide agents include monosaccharides, disaccharides, and other oligosaccharides with glass transition temperature of the maximally freeze-concentrated solution (Tg) that is at least −60, −50, −40, −30, −20, −10, or 0° C. An exemplary saccharide for use in cryopreservation is trehalose.

In some aspects, the three-dimensional tissues are treated to kill the cells prior to use. In some aspects, the ECM deposited on the scaffolds may be collected and processed for administration (see U.S. Pat. Nos. 5,830,708 and 6,280,284, incorporated herein by reference).

In other embodiments, the three-dimensional tissue may be concentrated and washed with a pharmaceutically acceptable medium for administration. Various techniques for concentrating the compositions are available in the art, such as centrifugation or filtering. Examples include, dextran sedimentation and differential centrifugation. Formulation of the three-dimensional tissues may also involve adjusting the ionic strength of the suspension to isotonicity (i.e., about 0.1 to 0.2) and to physiological pH (i.e., pH 6.8 to 7.5). The formulation may also contain lubricants or other excipients to aid in administration or stability of the cell suspension. These include, among others, saccharides (e.g., maltose) and organic polymers, such as polyethylene glycol and hyaluronic acid. Additional details for preparation of various formulations are described in U.S. Patent Publication No. 2002/0038152, incorporated herein by reference.

As discussed above, the ECM compositions of the present invention may be processed in a number of ways depending on the anticipated application and appropriate delivery or administration of the ECM composition. For example, the compositions may be delivered as three-dimensional scaffolds or implants, or the compositions may be formulated for injection as described above. The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The term “subject” as used herein refers to any individual or patient to which the subject methods are performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The ECM compositions of the present invention have a variety of applications including, but not limited to, promoting repair and/or regeneration of damaged cells or tissues, use in patches to promote tissue regeneration, use in tissue culture systems for culturing cells, such as stem cells, use in surface coatings used in association with implantable devices (e.g., pacemakers, stents, stent grafts, vascular prostheses, heart valves, shunts, drug delivery ports or catheters), promoting soft tissue repair, augmentation, and/or improvement of a skin surface, such as wrinkles, use as a biological anti-adhesion agent, as a biological vehicle for cell delivery or maintenance at a site of delivery or as an anti-inflammatory composition.

As discussed above, growth factors or other biological agents which induce or stimulate growth of particular cells may be included in the ECM and CCM compositions of the present invention. The type of growth factors will be dependent on the cell-type and application for which the composition is intended. For example, in the case of osteochondral cells, additional bioactive agents may be present such as cellular growth factors (e.g., TGF-β), substances that stimulate chondrogenesis (e.g., BMPs that stimulate cartilage formation such as BMP-2, BMP-12 and BMP-13), factors that stimulate migration of stromal cells to the scaffold, factors that stimulate matrix deposition, anti-inflammatories (e.g., non-steroidal anti-inflammatories), immunosuppressants (e.g., cyclosporins). Other proteins may also be included, such as other growth factors such as platelet derived growth factors (PDGF), insulin-like growth factors (IGF), fibroblast growth factors (FGF), epidermal growth factor (EGF), human endothelial cell growth factor (ECGF), granulocyte macrophage colony stimulating factor (GM-CSF), vascular endothelial growth factor (VEGF), cartilage derived morphogenetic protein (CDMP), other bone morphogenetic proteins such as OP-1, OP-2, BMP3, BMP4, BMP9, BMP11, BMP14, DPP, Vg-1, 60A, and Vgr-1, collagens, elastic fibers, reticular fibers, glycoproteins or glycosaminoglycans, such as heparin sulfate, chondroitin-4-sulfate, chondroitin-6-sulfate, dermatan sulfate, keratin sulfate, etc. For example, growth factors such as TGF-0, with ascorbate, have been found to trigger chondrocyte differentiation and cartilage formation by chondrocytes. In addition, hyaluronic acid is a good substrate for the attachment of chondrocytes and other stromal cells and can be incorporated as part of the scaffold or coated onto the scaffold.

Additionally, other factors which influence the growth and/or activity of particular cells may also be used. For example, in the case of chondrocytes, a factor such as a chondroitinase which stimulates cartilage production by chondrocytes can be added to the matrix in order to maintain chondrocytes in a hypertrophic state as described in U.S. Patent Application No. 2002/0122790 incorporated herein by reference. In another aspect, the methods of the present invention include the presence of polysulphated alginates or other polysulphated polysaccharides such as polysulphated cyclodextrin and/or polysulphated inulin, or other components capable of stimulating production of ECM of connective tissue cells as described in International Patent Publication No. WO 2005/054446 incorporated herein by reference.

As discussed above, the ECM compositions of the present invention may be processed in a variety of ways. Accordingly, in one embodiment, the present invention includes a tissue culture system. In various aspects, the culture system is composed of the ECM compositions described herein. The ECM compositions of the present invention may be incorporated into the tissue culture system in a variety of ways. For example, compositions may be incorporated as coatings, by impregnating three-dimensional scaffold materials as described herein, or as additives to media for culturing cells. Accordingly, in one aspect, the culture system can include three-dimensional support materials impregnated with any of the ECM compositions described herein, such as growth factors or embryonic proteins.

The ECM compositions described herein may serve as a support or three-dimensional support for the growth of various cell types. Any cell type capable of cell culture is contemplated. In one aspect, the culture system can be used to support the growth of stem cells. In another aspect, the stem cells are embryonic stem cells, mesenchymal stem cells or neuronal stem cells.

The tissue culture system may be used for generating additional ECM compositions, such as implantable tissue. Accordingly, culturing of cells using the tissue culture system of the present invention may be performed in vivo or in vitro. For example, the tissue culture system of the present invention may be used to generate ECM compositions for injection or implantation into a subject. The ECM compositions generated by the tissue culture system may be processed and used in any method described herein.

The ECM compositions of the present invention may be used as a biological vehicle for cell delivery. As described herein, the ECM compositions may include both soluble and/or non-soluble fractions. As such, in another embodiment of the present invention, a biological vehicle for cell delivery or maintenance at a site of delivery including the ECM compositions of the present invention, is described. The ECM compositions of the present invention, including cells and three-dimensional tissue compositions, may be used to promote and/or support growth of cells in vivo. The vehicle can be used in any appropriate application, for example to support injections of cells, such as stem cells, into damaged heart muscle or for tendon and ligament repair as described above.

Appropriate cell compositions (e.g., isolated ECM cells of the present invention and/or additional biological agents) can be administered before, after or during the ECM compositions are implanted or administered. For example, the cells can be seeded into the site of administration, defect, and/or implantation before the culture system or biological delivery vehicle is implanted into the subject. Alternatively, the appropriate cell compositions can be administered after (e.g., by injection into the site). The cells act therein to induce tissue regeneration and/or cell repair. The cells can be seeded by any means that allows administration of the cells to the defect site, for example, by injection. Injection of the cells can be by any means that maintains the viability of the cells, such as, by syringe or arthroscope.

In another embodiment, the present invention includes various implantable devices and tissue regeneration patches including the ECM compositions described herein which allow for benefits, such as tissue ingrowth. As discussed herein, the ECM compositions may serve as coatings on medical devices, such as patches or other implantable devices. In various aspects, such devices are useful for wound repair, hernia repair, pelvic floor repair (e.g., pelvic organ prolapse), rotator cuff repair and the like. In related aspects, coatings are useful for orthopedic implants, cardiovascular implants, urinary slings and pacemaker slings.

For example, the basic manifestation of a hernia is a protrusion of the abdominal contents into a defect within the fascia. Surgical approaches toward hernia repair is focused on reducing the hernial contents into the peritoneal cavity and producing a firm closure of the fascial defect either by using prosthetic, allogeneic or autogenous materials. A number of techniques have been used to produce this closure, however, drawbacks to current products and procedures include hernia recurrence, where the closure weakens again, allowing the abdominal contents back into the defect. In heriorrhaphy, a corrective tissue regeneration patch, such as a bioresorbable or synthetic mesh coated with ECM compositions could be used.

A variety of techniques are known in the art for applying biological coatings to medical device surfaces that may be utilized with the present invention. For example, ECM compositions may be coated using photoactive crosslinkers allowing for permanent covalent bonding to device surfaces upon activation of the crosslinker by applying ultraviolet radiation. An exemplary crosslinker is TriLite™ crosslinker, which has been shown to be non-cytotoxic, non-irritating to biological tissue and non-sensitizing. ECM materials may be unseparated or separated into individual components, such as human collagens, hyaluronic acid (HA), fibronectin, and the like before coating or incorporation into various implantable devices. Further, additional growth factors and such may be incorporated to allow for beneficial implantation characteristics, such as improved cell infiltration.

The compositions of the present invention may be prepared as known in the art, however employing the innovative culture methods described herein (e.g., culture under hypoxic conditions). The preparation and use of ECM compositions created under normal oxygen culture conditions for the repair and/or regeneration of cells, improvement of skin surfaces, and soft tissue repair are described in U.S. Pat. Nos. 5,830,708, 6,284,284, U.S. Patent Application No. 2002/0019339 and U.S. Patent Application No. 2002/0038152 incorporated herein by reference.

The compositions or active components used herein, will generally be used in an amount effective to treat or prevent the particular disease being treated. The compositions may be administered therapeutically to achieve therapeutic benefit or prophylactically to achieve prophylactic benefit. By therapeutic benefit is meant eradication or amelioration of the underlying condition or disorder being treated. Therapeutic benefit also includes halting or slowing the progression of the disease, regardless of whether improvement is realized.

The amount of the composition administered will depend upon a variety of factors, including, for example, the type of composition, the particular indication being treated, the mode of administration, whether the desired benefit is prophylactic or therapeutic, the severity of the indication being treated and the age and weight of the patient, and effectiveness of the dosage form. Determination of an effective dosage is well within the capabilities of those skilled in the art.

Initial dosages may be estimated initially from in vitro assays. Initial dosages can also be estimated from in vivo data, such as animal models. Animals models useful for testing the efficacy of compositions for enhancing hair growth include, among others, rodents, primates, and other mammals. The skilled artisans can determine dosages suitable for human administration by extrapolation from the in vitro and animal data.

Dosage amounts will depend upon, among other factors, the activity of the conditioned media, the mode of administration, the condition being treated, and various factors discussed above. Dosage amount and interval may be adjusted individually to provide levels sufficient to the maintain the therapeutic or prophylactic effect.

In one embodiment, the present invention presents a method for reducing inflammation in a subject comprising administering to the subject a conditioned culture medium (CCM) composition or an extracellular matrix (ECM) composition, wherein the composition is produced by culturing fibroblast cells on microcarrier beads or a three-dimensional surface under hypoxic conditions of 1-5% oxygen, thereby producing multipotent stem cells which produce a CCM and an ECM fraction, thereby reducing inflammation. In one aspect, the CCM composition is a soluble fraction. In another aspect, the ECM composition is a non-soluble fraction. In a further aspect, the inflammation is associated with bone degeneration, spinal disk degeneration, osteoarthritis, pruritis, skin inflammation, psoriasis, multiple sclerosis, rheumatoid arthritis, osteoarthritis, systemic lupus erythematosus, Hashimoto's thyroidis, myasthenia gravis, diabetes type I or II, asthma, inflammatory lung injury, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, inflammatory bowel disease, Crohn's disease, ulcerative colitis, an inflammatory disease of the joints, skin, or muscle, acute or chronic idiopathic inflammatory arthritis, myositis, a demyelinating disease, chronic obstructive pulmonary disease, interstitial lung disease, interstitial nephritis or chronic active hepatitis. In a specific aspect, the inflammation is associated with bone degeneration, spinal disk degenefration or osteoarthritis.

In an additional aspect, enzyme and protease activity in the anulus fibrosus (AF) and nucleus pulposus (NP) are decreased. In some aspects, the expression of IL-1, IL-6 and/or TNF in anulus fibrosus (AF) tissue is decreased and/or the expression of ACAN, MMP3 and/or ADAMTS4 is increased in AF tissue following administration of the CCM or the ECM. In certain aspects, the expression of TNF and/or MMP3 in nucleus pulposus (NP) tissue is decreased and/or expression of ADAMTS4 and/or ACAN in NP tissue following administration of the CCM or the ECM. In a further aspect, aggrecan is produced. In one aspect, the fibroblast cells are neonatal fibroblast cells.

Inflammation is part of the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. The classical signs of inflammation are heat, pain, redness, swelling, and loss of function.

There are several markers of inflammation, including IL-1, IL-6, TNF, MMP3, ADAMTS4 and ACAN. IL-1 plays a central role in the regulation of immune and inflammatory responses and expression IL-1 is associated with inflammation. IL-1 is produced by tissue macrophages, monocytes, fibroblasts, and dendritic cells, and is also expressed by B lymphocytes, NK cells, microglia, and epithelial cells. IL-1 acts to increase the expression of adhesion factors on endothelial cells to enable transmigration of immunocompetent cells, such as phagocytes, lymphocytes and others, to sites of infection.

IL-6 is an interleukin that acts as both a pro-inflammatory cytokine and an anti-inflammatory myokine. As such, expression of IL-6 is associated with inflammation. IL-6 is secreted by T cells and macrophages to stimulate immune response, e.g. during infection and after trauma, especially burns or other tissue damage leading to inflammation. IL-6 also plays a role in fighting infection. IL-6 stimulates the inflammatory and auto-immune processes in many diseases such as diabetes, atherosclerosis, depression, Alzheimer's Disease, systemic lupus erythematosus, multiple myeloma, prostate cancer, Behçet's disease, and rheumatoid arthritis.

TNF is a member of a superfamily and can cause apoptosis. TNF is a monocyte-derived cytotoxin that has been implicated in tumor regression, septic shock, and cachexia. TNF has a wide variety of functions including causing cytolysis of certain tumor cell lines; the induction of cachexia; it is a potent pyrogen, causing fever by direct action or by stimulation of interleukin-1 secretion; it can stimulate cell proliferation and induce cell differentiation under certain conditions. Increased TNF expression is associated with inflammation.

Proteins of the matrix metalloproteinase (MMP) family are involved in the breakdown of extracellular matrix proteins and during tissue remodeling in normal physiological processes, such as embryonic development and reproduction, as well as in disease processes, such as arthritis, and tumor metastasis. Matrix metalloproteinase 3 (MMP-3) degrades collagen types II, III, IV, IX, and X, proteoglycans, fibronectin, laminin, and elastin. In addition, MMP-3 can also activate other MMPs such as MMP-1, MMP-7, and MMP-9, rendering MMP-3 crucial in connective tissue remodeling. The enzyme is also thought to be involved in wound repair, progression of atherosclerosis, and tumor initiation. In addition, the activity of MMP3 in extracellular space, MMP3 can enter in cellular nuclei and control transcription.

A disintegrin and metalloproteinase with thrombospondin motifs 4 (ADAMTS4) is an enzyme that in humans is capable of cleaving all the large chondroitin sulfate hyaluronan-binding proteoglycans (CSPGs), including aggrecan, brevican, neurocan and versican and is inhibited by tissue inhibitor of metalloproteinase-3 (TIMP3). ADAMTS4 is responsible for the degradation of proteoglycans in articular cartilage in osteoarthritis.

Aggrecan core protein (ACAN) is an integral part of the extracellular matrix in cartilaginous tissue and it withstands compression in cartilage. Aggrecan is a critical component for cartilage structure and the function of joints. Aggrecan provides intervertebral disc and cartilage with the ability to resist compressive loads. The localized high concentrations of aggrecan provide the osmotic properties necessary for normal tissue function with the GAGs producing the swelling pressure that counters compressive loads on the tissue. This functional ability is dependent on a high GAG/aggrecan concentration being present in the tissue extracellular matrix. In the disc, aggrecan concentrations peak in a person's twenties, and decline thereafter, with aggrecan degradation products slowly accumulating over the following decades. This causes discs to get stiffer and less resilient with age.

In an additional embodiment, the present invention provides methods of treating intervertebrate disc degeneration (IDD) in a subject comprising administering to the subject a cell conditioned media (CCM) composition and/or an extracellular matrix (ECM) composition, wherein the CCM and ECM compositions are produced by culturing fibroblast cells on microcarrier beads or a three-dimensional surface under hypoxic conditions of 1-5% oxygen, thereby producing multipotent stem cells which produce a CCM and an ECM fraction, thereby treating IDD. In an additional aspect, enzyme and protease activity in the anulus fibrosus (AF) and nucleus pulposus (NP) are decreased. In some aspects, the expression of IL-1, IL-6 and/or TNF in anulus fibrosus (AF) tissue is decreased and/or the expression of ACAN, MMP3 and/or ADAMTS4 is increased in AF tissue following administration of the CCM or the ECM. In certain aspects, the expression of TNF and/or MMP3 in nucleus pulposus (NP) tissue is decreased and/or expression of ADAMTS4 and/or ACAN in NP tissue following administration of the CCM or the ECM. In one aspect, the fibroblast cells are neonatal fibroblast cells. In one aspect, aggrecan is produced. In an additional aspect, there is an increase in disc space and/or disc height. In a further aspect, there is a decrease in disc degeneration. In another aspect, hyaline cartilage and/or fibrous tissue is regenerated in the disc.

In a further embodiment, the present invention provides an anti-inflammatory composition which is a CCM composition or an ECM composition produced by culturing fibroblast cells on microcarrier beads or a three-dimensional surface under hypoxic conditions of 1-5% oxygen, thereby producing multipotent stem cells which produce a soluble and a non-soluble fraction and a pharmaceutically acceptable carrier.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

The ECM and CCM compositions will commonly comprise a solution of the peptide or peptide conjugate dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

Presented below are examples discussing generation of ECM compositions contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Preparation of CCM and hECM

Human neonatal fibroblasts were seeded on dextran beads and grown in a computer-controlled closed 10 L bio-reactor under 3-5% oxygen. Cells were fed a serum free media daily through a perfusion system. Once cells reached confluence the CCM was removed daily for up to twelve weeks, concentrated through a 10 kD filter, and clarified. Mass spectrometry analysis was performed by Proteome Sciences plc (UK) to identify and quantify proteins present in the CCM. The CCM samples underwent tryptic digest followed by liquid chromatography and mass spectroscopy followed by computational analysis to identify proteins present in the biological specimen.

The mass spectrometry data showed a composition consisting of over 100 soluble extracellular matrix proteins and growth factors in the unconcentrated and 10× concentrated CCM (Table 3). Further studies demonstrated excellent lot to lot consistency.

At the end of the 3-month culture period, the insoluble material consisting of microcarrier beads, cells and deposited ECM was collected, washed in sterile distilled water, and frozen at −80° C. The frozen insoluble material was thawed, washed twice in sterile PBS (Gibco, Grand Island, N.Y., USA) and mechanically homogenized (Polytron Kinematica, Luzern, Switzerland) and incubated with sterile-filtered dextranase (Sigma-Aldrich, St. Louis, Mo., USA) at 37° C. to digest the microcarrier beads. The solution was extensively washed with PBS to generate a final hECM material with a paste-like consistency and stored at 4° C. until used in experiments.

hECM material was biochemically characterized by measuring the collagen and sulfated glycosaminoglycan (sGAG) content. The final hECM material was frozen at −80° C. and lyophilized in a FreeZone 4.5 Liter benchtop freeze dry system (LabConco, Kansas City Mo.) to obtain dried material for collagen and sGAG analysis. The hydroxyproline content of lyophilized hECM was determined by first treating lyophilized hECM material with 6 N HCl and heating overnight at 115° C. The samples were neutralized to pH 7-8 with 1 N NaOH and then oxidized with Chloramine-T solution at room temperature followed by the addition of a perchloric acid solution. The samples were then reacted with p-dimethylaminobenzaldehyde (pDAB) and the colorimetric change was measured by at reading at 561 nm absorbance wavelength using a SpectraMax M3 plate reader (Molecular Devices, Sunnyvale, Calif.). The samples were fitted to a standard curve of 4-hydroxy-L-proline and the collagen content was calculated using a hydroxyproline concentration of 13.5% and expressed as mg collagen/mg dry weight of ECM.

sGAG content of lyophilized hECM was performed by enzymatically digesting the material overnight at 60° C. in a papain solution containing 0.1 M sodium phosphate, 10 mM EDTA solution, 5 mM cysteine solution, pH 6.5. The samples were reacted with the metachromatic dye dimethylmethylene blue (DMB) in a microtiter plate and read at an absorbance wavelength of 525 nm using a SpectraMax M3 plate reader (Molecular Devices, Sunnyvale, Calif.). The sample values were fitted to a standard curve of chondroitin sulfate from bovine cartilage and expressed as ug sGAG/mg dry weight of ECM. All chemicals for hydroxyproline and sGAG assays were from obtained from Sigma-Aldrich (St. Louis, Mo.).

The collagen and sGAG assay results confirmed lot-to-lot consistency of the material. Mass spec analysis showed that the hECM consists predominantly of human collagens I, II, III, and V, tenascin, vimentin, decorin, fibrillin, nexin, and basement membrane specific heparin sulfate proteoglycans.

TABLE 3

| Protein | | |
|---|---|---|
| Description | Protein Description | Protein Description |
| Serum albumin | Triosephosphate isomerase | Isoform 2 of Neuroblastoma |
| | | suppressor of tumorigenicity 1 |
| Serotransferrin | Complement Cis subcomponent | Isoform M1 of Pyruvate kinase |
| | | PKM |
| Isoform 15 of | Insulin-like growth factor- | Interleukin-8 |
| Fibronectin | binding protein 6 | |
| Collagen alpha-2(I) | Collagen alpha-1- (III) chain | Transforming growth factor-beta- |
| chain | | induced protein ig-h3 |
| Tenascin | Nucleobindin-1 | Isoform 3 of Latent-transforming |
| | | growth factor beta-binding |
| | | protein 1 |
| Collagen alpha-1(1) | Peptidylprolyl cis-trans | Isoform 2 of Fibulin-2 |
| chain | isomerase A | |
| Vimentin | Stromelysin-1 | Superoxide dismutase [Cu—Zn] |
| Follistatin-related | Isoform 2 of Mannan-binding | C-X-C motif chemokine 5 |
| protein 1 | lectin serine protease 1 | |
| Fibulin-1 | Cystatin-C | Prostaglandin-H2 D-isomerase |
| Lumican | Isoform 2 of Latent- transforming | Basement membrane-specific |
| | growth factor beta- binding | heparan sulfate proteoglycan core |
| | protein 4 | protein |
| Complement C3 | Keratin, type 1 cytoskeletal 9 | Filamin-A |
| Thrombospondin-1 | Laminin subunit beta-1 | Glyceraldehyde-3-phosphate |
| | | dehydrogenase |
| Metalloproteinase | Complement factor H | Isoform 2 of Insulin-like |
| inhibitor 1 | | growth factor-binding protein 3 |
| Insulin-like growth | Chitinase-3-like protein 1 | Trypsin-1 |
| factor-binding protein 4 | | |
| Collagen alpha-1- (VI) | Thymosin beta-4 | Aldose reductase |
| chain | | |
| Procollagen C- | Glia-derived nexin | Immunoglobulin superfamily |
| endopeptidase | | containing leucine-rich repeat |
| enhancer 1 | | protein |
| Interstitial | Sulfhydryl oxidase 1 | Retinoic acid receptor responder |
| collagenase | | protein 2 |
| Galectin-1 | Peptidylprolyl cis-trans | Cellular retinoic acid-binding |
| | isomerase B | protein 2 |
| Growth-regulated | Laminin subunit alpha-4 | Galectin-3 |
| alpha protein | | |
| 72 kDa type IV | Fatty acid-binding protein, heart | Tropomyosin alpha-4 chain |
| collagenase | | |
| Complement Clr | Interleukin-6 | Protein S100-A4 |
| subcomponent | | |
| Isoform 2 of Fructose- | Aminopeptidase N | Plasminogen activator inhibitor 1 |
| bisphosphate aldolase A | | |
| Collagen alpha-3- (VI) | Isoform 3 of L-lactate | Actin, cytoplasmic 1 |
| chain | dehydrogenase A chain | |
| Isoform 4 of Tenascin- | Beta-2-microglobulin | CD109 antigen |
| X | | |
| Isoform 2C2A of | Moesin | Phosphoglycerate kinase 1 |
| Collagen alpha-2- (VI) | | |
| chain | | |
| Prelamin-A/C | Olfactomedin-like protein 3 | Isoform 3 of Ectonucleotide |
| | | pyrophosphatase/phosphodiesterase |
| | | family member 2 |
| Tetranectin | Peroxiredoxin-1 | Isoform 2 of Glucose-6-phosphate |
| | | isomerase |
| SPARC | Phosphoglycerate mutase 1 | 78 kDa glucose-regulated protein |
| Extracellular matrix | Apolipoprotein D | Extracellular superoxide |
| protein 1 | | dismutase [Cu—Zn] |
| Gelsolin | Keratin, type I cytoskeletal 10 | Protein disulfide-isomerase A3 |
| Laminin subunit | Dickkopf-related protein 3 | CD59 glycoprotein |
| gamma-1 | | |
| Pigment epithelium- | 14-3-3 protein zeta/delta | Metalloproteinase inhibitor 2 |
| derived factor | | |
| Keratin, type ll | Phosphatidylethanolaniine- | Collagen alpha-1 (Xl1) chain |
| cytoskeletal 1 | binding protein 1 | |
| EGF-containing | Stanniocalcin-2 | Plasminogen activator inhibitor 2 |
| fibulin-like extra- | | |
| cellular matrix protein 1 | | |
| Insulin-like growth | Isoform 2 of Procollagen-lysine, | Galectin-3-hinding protein |
| factor-binding protein 5 | 2-oxaglutarate 5-dioxygenase 1 | |
| Stanniocalcin-1 | Fibrillin-1 | Sushi, van Wine-brand factor type |
| | | A, EGF and pentraxin domain- |
| | | containing protein 1 |
| Insulin-like growth | EM111N-1 | |
| factor-binding protein 2 | | |

### Example 2

### Cell Proliferation Assay

In vitro studies were performed to measure the ability of a human embryonic-like CCM to support mesenchymal stem cell (MSC) proliferation while maintaining a stem phenotype. Human bone marrow MSCs were obtained from Cellular Engineering Technologies, Coralville, Iowa, Cat #HMSC-BM100 and cultured in monolayer with Hyclone Advance STEM medium (Hyclone Catalog SV30110.01) with growth supplements and 1% antibiotic/antimycotic additive. Cells were cultured for 3 days, passaged and then plated in T25 flasks. Control cultures received total growth medium while test cultures were fed with growth medium supplemented with 10% CCM. Cell counts were done at day 6 and 13.

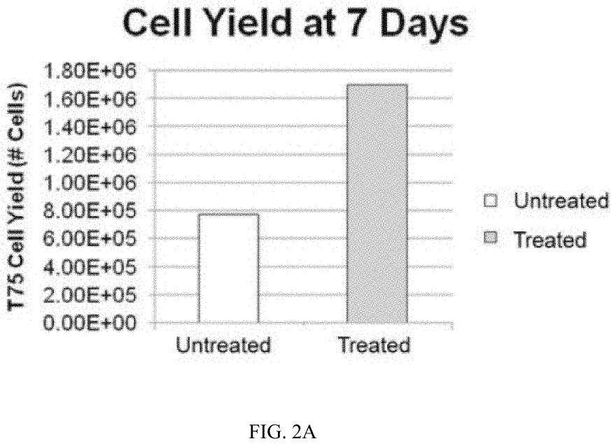

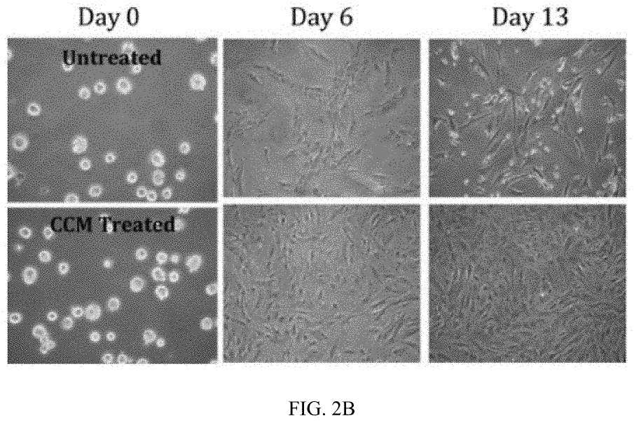

Cell counts at day 7 after treatment with CCM supplemented media showed that the treated cultures had more than twice the number of MSCs. Enhanced proliferation of the treated cells continued to be seen past day 7 (FIG. 2A) the morphology of the cells was normal and treated cells reached confluence by day 13 whereas control cultures were less than 50% confluent at the same time (FIG. 2B).

### Example 3

### Ex Vivo Rabbit Disc Study

An ex vivo thrombin-induced inflammation rabbit disc model was utilized to study the ability of CCM and hECM to reduced inflammatory factors and protease activity. New Zealand White Rabbit spines were isolated and cut into bone-disc-bone segments and cultured in nutrient media for 24 hours. Discs were then injected with 10 ul of thrombin to induce a severe inflammatory reaction. After 24 hours the AP and NP areas of the disc were injected with either 10 ul of phosphate buffered saline, hECM or CCM. Analysis of upregulated genes was assessed at 24 and 48 hours by qPCR.

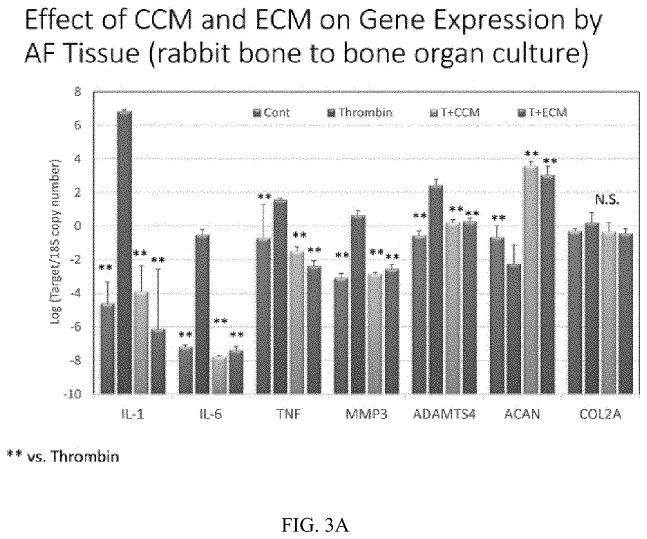

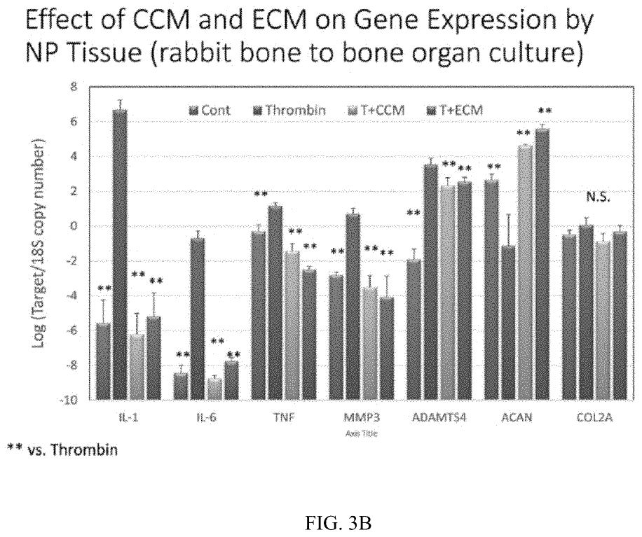

In hECM and CCM treated enzyme and protease activity was significantly decreased after only 24 hours, with levels being reduced to baseline or below (p<0.0001). In addition, the treated IVDs demonstrated stimulation of the native disc cells to make new aggrecan (p<0.0001), a critical component in both the AF and NP of the disc (FIG. 3). Given that the pathophysiology degeneration is associated with increased localized inflammation and enzymatic degradation and a breakdown of matrix proteins having biomaterial that can reverse these localized effects has the potential of offering therapeutic benefit.

### Example 4

### In Vivo Rabbit Disc Study

New Zealand White rabbits [specific pathogen-free (SPF), 4-6 months-old, Western Oregon Rabbit Co. Philomath, Oregon] were used. Two to five days before the surgery, a pre-operative X-ray was taken as a baseline control under isoflurane anesthesia. Animals received Buprenex SR (0.1 mg/kg) or Fentanyl Patch (12 mg) preoperatively as a preemptive analgesic. A dose of ketamine hydrochloride (35 mg/kg) xylazine (5 mg/kg) was given intramuscularly. Animals were intubated and maintained by isoflurane inhalation (induced at 2-3%, and maintained at 1-4%). The rabbits received 50 cc of fluids (Lactated Ringer's solution or NaCl) SQ while being prepped for surgery. Following prepping and draping, the lumbar IVDs were exposed through a posterolateral retroperitoneal approach by blunt dissection of the psoas muscle. The anterior surfaces of three consecutive lumbar IVDs (L2/3, L3/4 and L4/5) were exposed. Using an 18 G needle with a stopper device that allows the needle to penetrate to a depth of 5 mm, the AF were punctured in the ventral aspect into the NP at the L2/3 and L4/5 levels. A titanium staple and a black silk suture were placed at L3/4 level as a reference point. The surgical wounds created were repaired in layers and the skin was closed using staples. During or after the surgery, an intra-operative X-ray was taken to confirm the level of puncture.

Two weeks after the initial surgery (annular puncture), a similar surgical procedure was made from the opposite side to avoid bleeding from the scar formed from the first operation. Once the surgically degenerating discs were confirmed by X-ray and visual inspection, test and control materials were intradiscally injected into the NP area with a microsyringe with 23 G needle at both the L2/3 and L4/5 levels for each rabbit. At 2, 4, and 6 weeks after the initial annular puncture, an X-ray to measure IVD height was taken. X-rays were taken under isoflurane anesthesia.

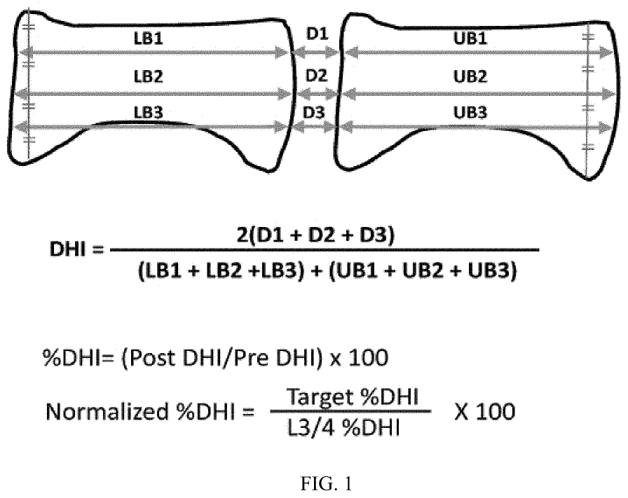

All X-ray images were independently interpreted by an orthopedic researcher who was blinded to the study group and time point. Using digitized X-rays, measurements, including the vertebral body height and IVD height, were analyzed using the custom program for MATLAB software (Natick, Mass.). Data were transported to Excel software and the IVD height expressed as the disc height index (DHI=IVD height/adjacent IVD body height) based on the previously developed method. The mean IVD height (DHI) was calculated by averaging the measurements obtained from the anterior, middle and posterior portions of the IVD and dividing that by the mean of the adjacent vertebral body heights. The average percent change in DHI of injected discs (both L2/3 and L4/5) was calculated for each postoperative disc as a ratio to its preoperative DHI (% DHI=postoperative DHI/preoperative DHI×100) and further normalized to the DHI of the non-punctured disc (L3/4): Normalized % DHI=(punctured % DHI/non-punctured % DHI×100). (FIG. 1) All radiographs were assessed by an observer blinded to this experiment.

MRI examinations were performed on all rabbit discs after sacrifice. MRI examinations on dissected specimens were performed on all the rabbits in the study using a BioSpec 70/30 USR (Bruker, ON). After sacrifice, the spinal columns, with surrounding soft tissue, were isolated and imaged. For anatomic images and Pfirrmann grading, T2-weighted images in the sagittal plane were obtained using the following settings: fast spin echo sequence with TR (time to repetition)=3000 ms, TE (time to echo)=100 ms, matrix=512×256, FOV=10, NEX=8, and slice thickness=1 mm. Fifteen slices were obtained with a 0 mm gap. For semi-quantitative morphologic assessment, T2-weighted images were evaluated by two observers blinded to the experiment using the Pfirrmann classification based on changes in the degree and area of signal intensity from grades 1-5. The grades from the three observers were averaged to represent the grade for each disc. Histological processing was conducted to assess tissue quality of control, non-punctured, and treated disc tissue.

Midsagittal sections (5 rim) of each experimental IVD from rabbits sacrificed at 6 weeks were stained with either hematoxylin and eosin or safranin-O. An observer blinded to this experiment analyzed the histologic sections and graded them using an established protocol (Table 4).

Histological grading scale based on four categories of degenerative changes with scores ranging from a normal disc with 4 points (1 point in each category) to a severely degenerated disc with 12 points (3 points in each category).

TABLE 4
I. Annulus Fibrosus:
Grade:

| | |
|---|---|
| 1. | Normal, pattern of fibrocartilage lamellae (U-shaped in |
| | the posterior aspect and slightly convex in the anterior |
| | aspect) without ruptured fibers and without a |
| | serpentine appearance anywhere within the annulus |
| 2. | Ruptured or serpentined patterned fibers in less |
| | than 30% of the annulus |
| 3. | Ruptured or serpentined patterned fibers in more |
| | than 30% of the annulus |

II. Border between the annulus fibrosus and nucleus pulposus:
Grade:

| | |
|---|---|
| 1. | Normal |
| 2. | Minimally interrupted |
| 3. | Moderate/severe interruption |

III. Cellularity of the nucleus pulposus:
Grade:

| | |
|---|---|
| 1. | Normal cellularity with large vacuoles in the |
| | gelatinous structure of the matrix |
| 2. | Slight decrease in the number of cells and fewer vacuoles |
| 3. | Moderate/severe decrease (>50%) in the number of cells |
| | and no vacuoles |

IV. Matrix of the nucleus pulposus:
Grade:

| | |
|---|---|
| 1. | Normal gelatinous appearance |
| 2. | Slight condensation of the extracellular matrix |
| 3. | Moderate/severe condensation of the extracellular matrix |

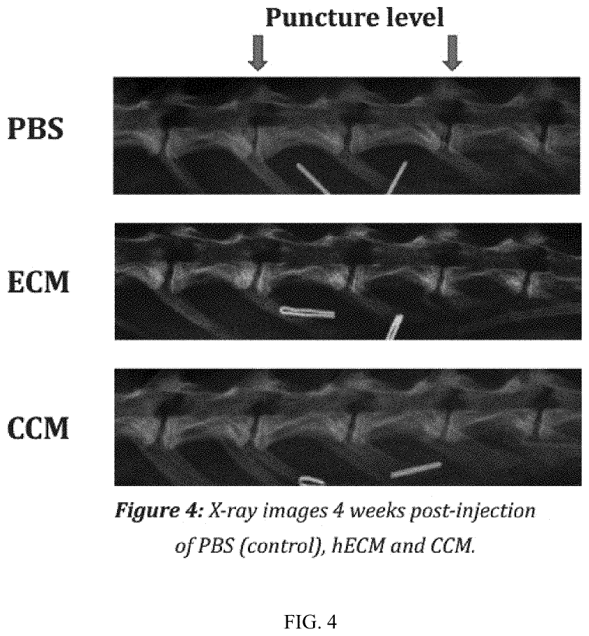

The initial annular injury was induced in rabbit discs of 18 NZW rabbits; this procedure resulted in degenerative changes in the NP and AF. After 2 weeks, the rabbits were then injected with a vehicle control, hECM or CCM and followed up by X-ray. At 4 weeks after injection, both the hECM and CCM group showed wider disc space at the puncture level compared to the PBS control group (FIG. 4).

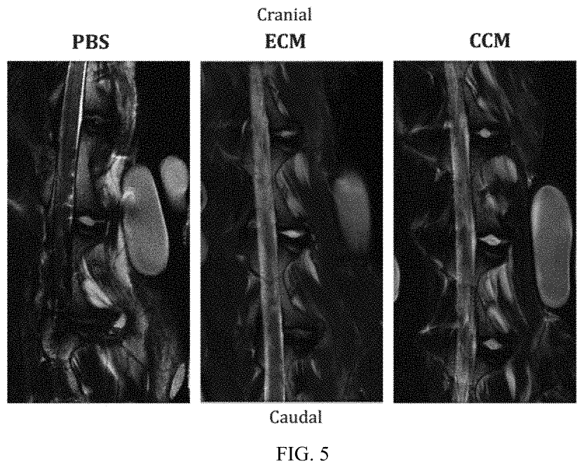

The ECM and CCM group showed a tendency to be higher in disc height, compared to the PBS group, at the 4-week post-injection time point (p=0.09 and p=0.10, respectively, FIG. 5).

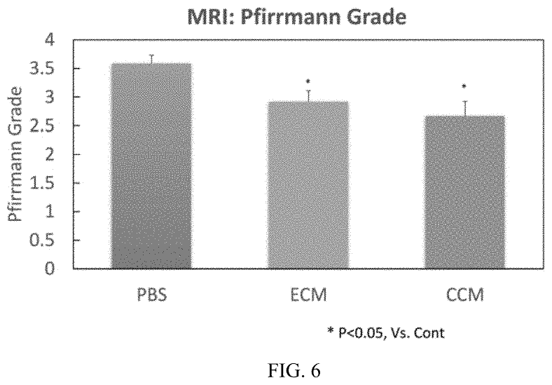

Four weeks after PBS, hECM or CCM injection, L3/4 control discs in all groups did not exhibit disc degeneration (FIG. 5). The signal intensity of NP in the CCM and ECM groups was stronger than in the PBS group. (FIG. 6, center and right panel).

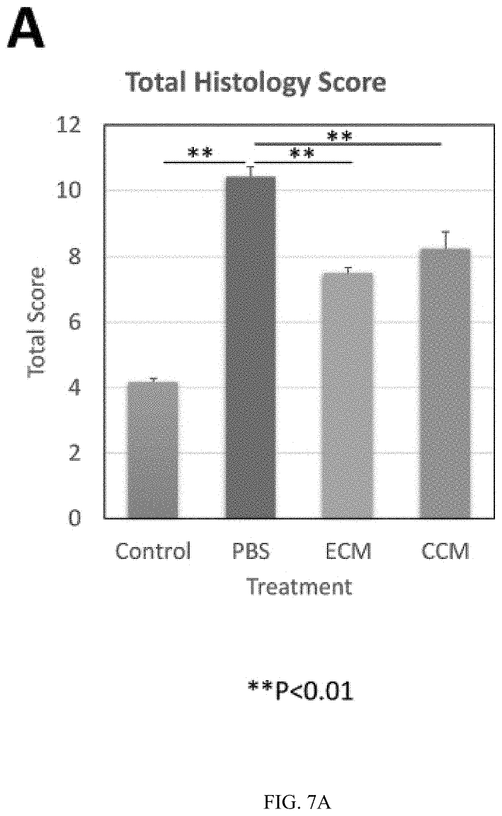

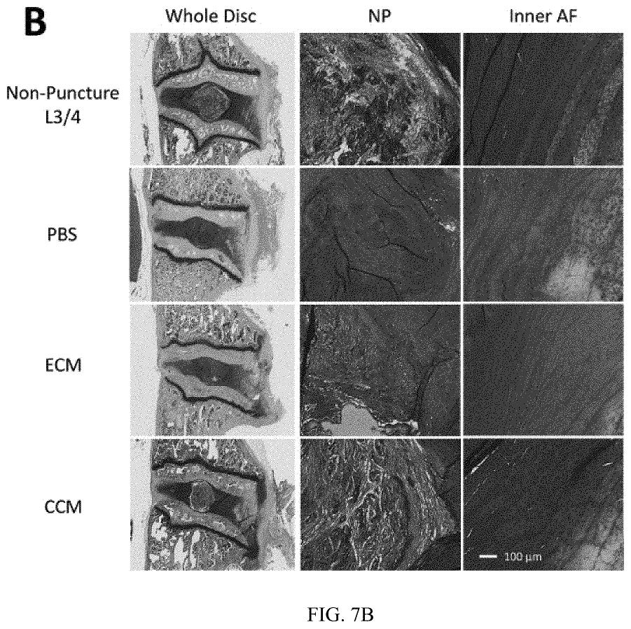

After 4 weeks following treatment, there was a significant difference between PBS vs. treatment groups in Pfirrmann Grading (p<0.05, FIG. 7).

Total histological scores of all puncture discs were significantly higher than that of the non-puncture control discs (L3/4). The injection of PBS did not show a significant effect on the overall histological score of disc degeneration 4 weeks after injection. The score in the CCM and hECM groups were significantly higher than in the PBS group (both P<0.01). The NP in CCM showed the maintenance of the original structure and the cells in the AF show round-shape chondrocytic changes. In the hECM group, a similar trend was seen.

The results from this study support further investigation into using the bioengineered biomaterials as a treatment to regenerate new functional tissue in intervertebral disc degeneration which was either caused by a recent traumatic accident or damage due to inflammatory cytokines and matrix degradation by local protease activity. A treatment with anti-inflammatory and regenerative properties, like those seen with both CCM and hECM, has the potential to be utilized at the time of IVD injury or in conjunction with inflammation induced IDD to heal/regenerate hyaline cartilage and fibrous tissue and prevent further matrix degeneration, resulting in more rapid and full recovery. In addition to reducing/eliminating harmful protease and inflammatory cytokine activity both biomaterials stimulate native cells in the disc to result in the maintenance of disc tissue, formation of new matrix and partially restored disc height in as little as 4 weeks. Given that the CCM demonstrated the most significant regeneration of tissue and is easily injected into the con-fined area of a damage disc, future long-term animal studies and clinical studies will focus on the use of the soluble material for disc repair and regeneration.

### Example 5

### Nucleus Pulposus Cell-Based Bioassay

A nucleus pulposus cell-based assay was used to assess biological activity of the concentrated cell-conditioned medium (cCCM) formulations described herein for the treatment of intervertebral disc degeneration (IVDD). The principle of this biopotency assay was based on measuring the physiological response, as measured by the degree of proliferation of cultured human nucleus pulposus cells (NPCs) upon stimulation with a test article. Cell proliferation was measured after incubation with test articles using colorimetric metabolic dyes to assess bioactivity.

The nucleus pulposus (NP) is the innermost part of the intervertebral disc (IVD) and is characterized biochemically as an extracellular matrix-rich tissue compartment containing collagen type II and sulfated glycosaminoglycans. Degeneration of the NP induced by a variety of causes such as trauma, aging or genetic and environmental factors triggers a cascade of inflammation, ECM catabolism and altered NPC phenotypes in which cell senescence, death and reduction of ECM production causes a disruption in the homeostasis of the tissue. Eventually, degradation of the NP can lead to mechanical instability of the IVD with the potential for prolapse or extrusion of the NP through the surrounding annulus fibrosis occurring which can cause destruction of the IVD with associated lack of mobility and increased pain. Stimulating the proliferation and self-renewal of the population of NPCs is crucial to inhibiting further degradation of the NP and restoring IVD health.

Cryopreserved primary human NPCs were cultured using a commercially available NP-specific complete medium as the growth medium to expand the NPCs in standard monolayer cell culture configuration. The test article was serially diluted beginning with an assay medium (assay medium consisted of a DMEM/F-12 containing 10% FBS, L-glutamine and antibiotics/antimycotic) to form a 12-point concentration curve in a 96-well multiwell plate. The NPCs were then harvested via trypsinization and seeded into the multiwell plate at a density of 2,500 cells/well. The culture was incubated for a duration of 68 hours at 37° C./5% CO₂. After the culture period, the medium was removed and replaced with assay medium containing 10% resazurin and incubated for 5 hours. Metabolic activity converts the resazurin dye to a fluorescent derivative that can be measured spectrophotometrically and is correlated to the number of cells present in the culture. The resulting dose-response curve for the test articles were fitted to a 4-parameter logistic (4-PL) regression curve resulting in a characteristic sigmoidal dose-response profile. The inflexion point of the regression curve, defined by the “C” coefficient of the 4-PL, was used to calculate the 50% effective concentration (EC50) of the test article. The EC50 of the test article can be used as a reportable value of the biopotency assay independent of a reference standard. However, the original data was transformed using the plate reader software program (SoftMax, Molecular Devices) in which the parameters of the regression lines of a reference standard, such as a recombinant growth factor or small molecule reagent, and test article were fitted to a constrained model allowing for parallel line analysis (PLA). The test article was assigned a relative potency value based on the reference standard value set to a value of “1.0”. The PLA method minimizes inter-assay variability due to changes in assay conditions and is an accepted form of reporting [CGI] a value for the bioactivity of the unknown test article. Standard operating procedures, forms and material specifications have been generated in regulatory support of the assay under cGMP. Assay development, qualification and validation was directed by regulatory guidance documents provided for industry.

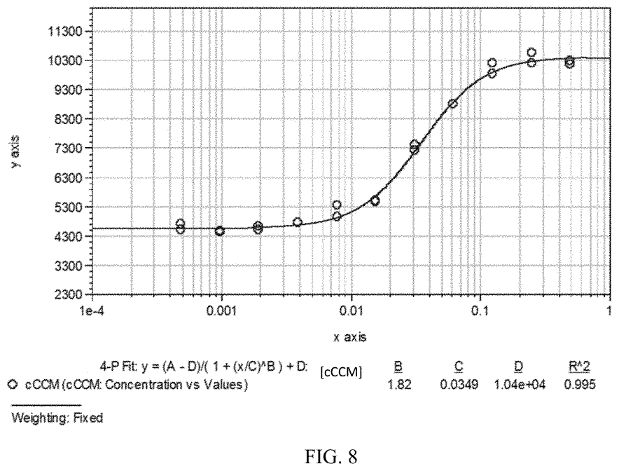

NPCs respond to cCCM treatment. FIG. 8 shows NPCs proliferating in a dose-dependent manner upon treatment with a cCCM formulation. Cell number increased upon exposure to increasing concentrations of cCCM which is indicated by an increase in fluorescent signal that can be positively correlated visually to the degree of cell proliferation. The observed dose-response treatment effect of cCCM to cultured demonstrates the functional ability of the assay to detect a stimulus effect of the test article.

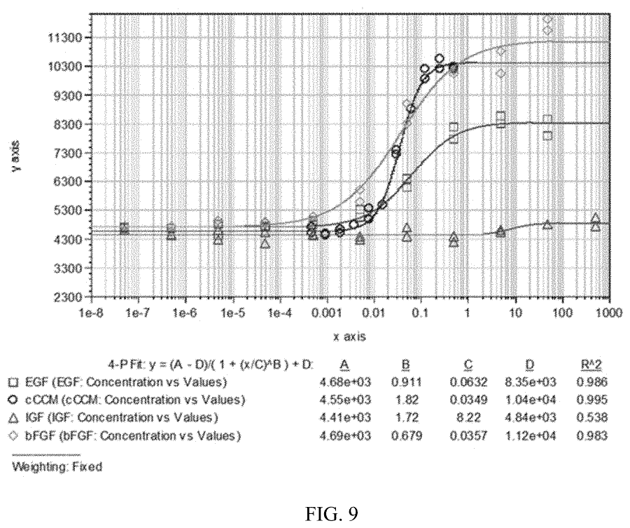

NPC assay can detect differential activity amongst sample types. FIG. 9 shows the proliferative effect of different recombinant human growth factors and cCCM in the NPC assay. Recombinant proteins used as test articles induced separate levels of efficacy and potency. Treatment of the NPCs with human bFGF, EGF and IGF-1 resulted in dose-dependent proliferation in all three cases, however with different levels of efficacy as indicated by the upper asymptote of the individual protein dose-response profiles. This demonstrates the ability of the assay to detect differences in the bioactivity of test articles and further confirms the value of the assay to serve as an indicator of biological potency.

NPC biopotency assay demonstrates an ability to identify biological activity in test articles by inducing the proliferation of the cells in a controlled, in-vitro multiwell format. The advantage of this assay is that it is multi-modal in approach and does not rely on the activity of specific molecular or cell-signaling pathways, but rather demonstrates biopotency via a positive physiological response of the cell as a whole, the proliferation of a population of nucleus pulposus cells isolated from human IVDs, to evaluate the potency of test articles.

Although the invention has been described with reference to the above examples, it will be understood that modifications and variations are encompassed within the spirit and scope of the invention. Accordingly, the invention is limited only by the following claims.

What is claimed is:

1. A method for reducing inflammation in a subject comprising administering to the subject a conditioned culture medium (CCM) composition or an extracellular matrix (ECM) composition, wherein the CCM and ECM compositions are produced by the method comprising culturing fibroblast cells on microcarrier beads or a three-dimensional surface under hypoxic conditions of 1-5% oxygen, thereby producing multipotent stem cells which produce a CCM and an ECM fraction, thereby reducing inflammation.

2. The method of claim 1, wherein the inflammation is associated with bone degeneration, spinal disk degeneration, osteoarthritis, pruritis, skin inflammation, psoriasis, multiple sclerosis, rheumatoid arthritis, osteoarthritis, systemic lupus erythematosus, Hashimoto's thyroidis, myasthenia gravis, diabetes type I or II, asthma, inflammatory lung injury, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, inflammatory bowel disease, Crohn's disease, ulcerative colitis, an inflammatory disease of the joints, skin, or muscle, acute or chronic idiopathic inflammatory arthritis, myositis, a demyelinating disease, chronic obstructive pulmonary disease, interstitial lung disease, interstitial nephritis or chronic active hepatitis.

3. The method of claim 2, wherein the inflammation is associated with bone degeneration, spinal disk degeneration or osteoarthritis.

4. The method of claim 1, wherein enzyme and protease activity in the anulus fibrosus (AF) and nucleus pulposus (NP) are decreased.

5. The method of claim 1, wherein the expression of IL-1, IL-6 and/or TNF in anulus fibrosus (AF) tissue is decreased following administration of the CCM or the ECM.

6. The method of claim 1, wherein the expression of ACAN, MMP3 and/or ADAMTS4 is increased in AF tissue following administration of the CCM or the ECM

7. The method of claim 1, wherein the expression of TNF and/or MMP3 in nucleus pulposus (NP) tissue is decreased following administration of the CCM or the ECM.

8. The method of claim 1, wherein the expression of ADAMTS4 and/or ACAN in NP tissue are increased following administration of the CCM or the ECM.

9. The method of claim 1, wherein aggrecan is produced.

10. The method of claim 1, wherein the fibroblast cells are neonatal fibroblast cells.

11. A method of treating intervertebrate disc degeneration (IDD) in a subject comprising administering to the subject a cell conditioned media (CCM) composition and/or an extracellular matrix (ECM) composition, wherein the CCM and ECM compositions are produced by the method comprising culturing fibroblast cells on microcarrier beads or a three-dimensional surface under hypoxic conditions of 1-5% oxygen, thereby producing multipotent stem cells which produce a CCM and an ECM fraction, thereby treating IDD.

12. The method of claim 11, wherein the expression of IL-1, IL-6 and/or TNF in anulus fibrosus (AF) tissue is decreased following administration of the CCM or the ECM.

13. The method of claim 11, wherein the expression of ACAN, MMP3 and/or ADAMTS4 is increased in AF tissue following administration of the CCM or the ECM.

14. The method of claim 11, wherein the expression of TNF and/or MMP3 in nucleus pulposus (NP) tissue is decreased following administration of the CCM or the ECM.

15. The method of claim 11, wherein the expression of ADAMTS4 and/or ACAN in nucleus pulposus (NP) tissue is increased following administration of the CCM or the ECM.

16. The method of claim 11, wherein the fibroblast cells are neonatal fibroblast cells.

17. The method of claim 11, wherein there is an increase in disc space and/or disc height.

18. The method of claim 11, wherein there is a decrease in disc degeneration.

19. The method of claim 11, wherein aggrecan is produced.

20. The method of claim 11, wherein hyaline cartilage and/or fibrous tissue is regenerated in the disc.

21. An anti-inflammatory composition comprising a CCM composition or an ECM composition produced by the method comprising culturing fibroblast cells on microcarrier beads or a three-dimensional surface under hypoxic conditions of 1-5% oxygen, thereby producing multipotent stem cells which produce a soluble and a non-soluble fraction and a pharmaceutically acceptable carrier.
